(19) United States
(12) Patent Application Publication
(10) Pub. No.: US 2005/0089924 A1
(43) Pub. Date: Apr. 28, 2005

(54) Biosensors and methods for their use

(76) Inventors: Chih-Ming Ho, Brentwood, CA (US); Tza-Huei Wang, Los Angeles, CA (US)

Correspondence Address: William J Wood;Gates & Cooper, Suite 1050, 6701 Center Drive West, Los Angeles, CA, 90045, US

(21) Appl. No.: 10/332,606

(22) PCT Filed: Aug. 14, 2001
(86) PCT No.: PCT/US01/25444

Related U.S. Application Data
(62) Division of application No. 60/225,077, filed on Aug. 14, 2000.

(51) Int. Cl. C12Q 1/68; G01N 33/53; C12M 1/34

(52) U.S. Cl. 435/7.1; 435/287.2; 435/6

(57) ABSTRACT

The invention disclosed herein provides biosensors and methods which increase the sensitivity of assays of optically labelled molecules fluorescently tagged polypeptides and polynucleotides while decreasing the sample volume required for detection. By integrating reflective sidewalls into the receptacles used in such assays, the signal-to-noise ratio of the optical signal is increased significantly. Typically the receptacles are microchannels. In addition, the geometry of the receptacles can be controlled C to further optimize the signal-to-noise ratio of the optical signal. The invention disclosed herein further provides methods and devices involving integrated electronics, wherein an element such as a diode, a transistor, an integrated circuit etc., is integrated with a bioreactor/channel in order to facilitate the detection or fabrication of bio-materials.

This application claims the benefit of U.S. provisional application No. 60/225,077, filed Aug. 14, 2000, the entire contents of which are incorporated herein by reference.

This invention was made with Government support under Grant No. N66001-96-C-83632 awarded by the Navy. The Government has certain rights in this invention.

### FIELD OF THE INVENTION

The invention described herein relates to biosensors for the detection of biological molecules such as polynucleotides. The invention further relates to methods and devices involving integrated electronics, wherein an element such as a diode, a transistor, an integrated circuit etc., is integrated with a bio-reactor/channel in order to facilitate the detection and/or fabrication of bio-materials.

### BACKGROUND OF THE INVENTION

Biosensors are sensors that detect chemical species with high selectivity on the basis of molecular recognition rather than the physical properties of analytes. See, e.g., Advances in Biosensors, A. P. F. Turner, Ed. JAI Press, London, (1991). Many types of biosensing devices have been developed in recent years, including enzyme electrodes, optical immunosensors, ligand-receptor amperometers, and evanescent-wave probes. Updike and Hicks, Nature, 214: 986 (1967), Abdel-Latif et al., Anal Lett., 21: 943 (111988); Giaever, J. Immunol., 110: 1424 (1973); Sugao et al. Anal. Chem, 65: 363 (1993), Rogers et al. Anal. Biochem., 182: 353 (1989).

DNA hybridization and immunoassay (e.g. ELISA) are typical sensor based methods for identifying of biological agents with high specificity. The typical processes to accomplish the identification comprise immobilizing a molecular probe, DNA or antibody, on the sensor surface, capturing the target molecules prepared from bio-agents onto the surface via specific DNA(probe)-DNA(target) hybridization or antibody(probe)-antigen(target) binding, applying secondary probes modified with either fluorescence or enzyme to bind to the target molecules for either optical or electrical signal detection, and then washing the non-binding molecules (probes, enzyme or substrate) away to reduce noise.

Because DNA/RNA analysis plays an extremely important and fundamental role in the rapid development of molecular diagnostics, genetics, and drug discovery, such analyses are of particular interest to practitioners in this field. One of the fastest growing areas in DNA/RNA analysis is the development of DNA-based biosensors. A variety of biosensors, both optical and electrochemical, have been developed for gene sequence analysis and biological pathogen detection [See, e.g., M. Yang, et al., Analytica Chimica Acta, 346(1997), 259-275; D. Ivnitski et al., Biosensors & Bioelectronics 14 (1999), 599-624] based on the DNA hybridization technique. In DNA hybridization, the target gene sequence is identified by a DNA probe that can form a double-stranded hybrid with its complementary nucleic acid with high efficiency and extremely specificity.

Typical DNA hybridization based biosensors require the steps of immobilization of DNA probes on the sensor surface and washing away the non-specific molecule binding to ensure specificity. The non-perfect surface modification from immobilization and incomplete washing are the main sources of noise and hence determine the ultimate sensitivity of the assays [see, e.g., Y. F. Chen et al., The 3^{rd} International conference on the interaction of Art and Fluid Mechanics, Zurich, Switzerland, 2000; J. Gau et al., Proceedings of the Fourth International Symposium on μ-TAS (2000), 509-512]. To immobilize probe molecules on the sensor surface and to achieve efficient washing require more fluidic devices to deal with excessive solutions if automation is desired. These are also time and power consuming steps. In addition, the immobilized monolayer can be destroyed by a high temperature condition that limits the post fabrication (chip bonding) choices if a closed sensor is to be developed. All of these issues, which come from these cumbersome steps, add complexities to the lab-on-chip design.

As is known in the art, molecular beacon (NM) based RNA-DNA hybridization techniques can be used to detect polynucleotides without immobilization and washing steps [See, e.g., XY Liu et al., Anal Biochem. 283(2000), 56-63; S. K Poddar et al., J. Virological Methods 82 (1999), 19-26; T. H Wang et al, proceedings of METMBS'00, pp295-300]. Molecular beacon technology utilizes oligonucleotide probes that become fluorescent only upon hybridization with target DNA/RNA molecules. By using this technique, the biosensor can provide high specificity without having to wash away the excess non-hybridized probes which are not fluorescent (if they exist in the solution).

With the introduction of molecular beacon technology, DNA detection with high specificity can be performed directly in a microchannel. (inchannel detection) which reduces the necessary sample volume by several orders of magnitude compared with most of the other DNA sensors [See, e.g., Y. F. Chen et al., The 3^{rd} International conference on the interaction of Art and Fluid Mechanics, Zurich, Switzerland, 2000; J. Gau et al, Proceedings of the Fourth International Symposium on μ-TAS (2000), 509-512] and further simplifies the processes of integrating a biosensor into a micro total analysis system(μ-TAS).

As noted above, the cumbersome steps in conventional polynucleotide detection methods add complexities to lab-on-chip design and create a number of other limitations including imperfect surface modification in immobilization and incomplete washing, both of which are the main sources for non-specific signal noise and hence influence the ultimate sensitivity of such assays. Consequently there is a need in the art for additional devices and methods for performing such assays in order to overcome these limitations in the art accepted methods. The invention described herein meets that need.

### SUMMARY OF THE INVENTION

The invention disclosed herein provides biosensors and methods which increase the sensitivity of assays of optical assays while decreasing the sample volume requited for detection. In particular, by integrating sidewall mirrors into microchannels used in assays of fluorescently tagged molecules, the signal-to-noise ratio of the fluorescent signal can be increased significantly. In addition, the geometry of the microchannels can be controlled to further optimize the signal-to-noise ratio of the fluorescent signal.

The invention disclosed herein has a number of embodiments. A preferred embodiment of the invention is a biosensor comprising a microchannel, wherein a sidewall of the microchannel has been treated so as to reflect a fluorescence signal such that the signal-to-noise ratio of the reflected fluorescence signal is increased. In such embodiments the microchannel can be treated to reflect a fluorescence signal by coating the sidewall with a reflective film of a metal such as gold or aluminum. In such embodiments, the signal-to-noise ratio of the reflected fluorescence signal can be enhanced by about 14% to about 420% and from about 80% to about 860% respectively, for different concentrations of a sample solution.

In highly preferred embodiments of the invention, the cross-section geometrical shape of the microchannel is selected to enhance the reflected signal-to-noise ratio. In representative embodiments, the microchannel has a cross-section geometrical shape selected from the group consisting of a rhombus, a trapezoid, a v-groove and a rectangle. In highly preferred embodiments, the microchannel has a cross-section geometrical shape that is trapezoidal.

As disclosed herein, the biosensor comprising a microchannel can be fabricated by any one of a variety of techniques known in the art such as KOH etching. In addition, the biosensor with the microchannel can be fabricated on to any one of the wide variety of matrices known in the art such as a microchip.

Related embodiments of the invention include a method of measuring a fluorescence signal comprising measuring the signal of a fluorescent molecule within a microchannel, wherein a sidewall of the microchannel has been treated so as to reflect the fluorescence signal such that the signal-to-noise ratio of the reflected fluorescence signal is increased. In such methods the microchannel can be treated to reflect a fluorescence signal by coating the sidewall with a reflective film of a metal. In addition, in such methods the geometry of the microchannel can be is selected to enhance the reflected signal-to-noise ratio. In preferred embodiments of this method, the fluorescence signal is measured by a laser induced fluorescence system.

Yet another embodiment of the invention includes a method of enhancing the optical measurement of a fluorescent signal of a fluorophore coupled to a polynucleotide or a polypeptide, the method comprising measuring the fluorescent signal of the fluorophore coupled molecule within a microchannel, wherein a sidewall of the microchannel is treated so as to reflect the fluorescence signal such that the signal-to-noise ratio of the reflected fluorescence signal is increased. In such methods the microchannel can be treated to reflect a fluorescence signal by coating the sidewall with a reflective film of a metal. In addition, in such methods the geometry of the microchannel can be is selected to enhance the reflected signal-to-noise ratio.

In preferred embodiments of this method, the fluorophore coupled molecule is a polynucleotide, for example a molecular beacon probe having a 5′ end labeled with a fluorescein moiety and a 3′ end labeled with a fluorescein quenching moiety. In preferred embodiments of this method, the fluorescence signal is measured by a laser induced fluorescence system. In one embodiment of this method, the volume of a media having the fluorophore coupled molecule is less than about 50 picoliters. In a specific embodiment of this invention, the molecular beacon probe is used to detect DNA. In highly preferred embodiments of the invention, the concentration of DNA detected is less than about 0.1 zmol.

The invention disclosed herein is further directed to integrated electronics, wherein an electronic element such as a diode, a transistor, an integrated circuit etc., is integrated with a bio-reactor/channel in order to facilitate the detection or fabrication of bio-materials. The disclosure provided herein allows one to perform DNA, antibody or other biological molecule detection without using two of the major and necessary steps of traditional detection methods, immobilization and washing.

The methods and devices disclosed herein have a number of embodiments which provide innovative approaches to the detection of various macromolecules by, for example, separating the DNA hybridization/immunobiological binding process and the enzymatic reaction process for sensing into two locations by applying an electrophoretic separator. The excess enzymes and other unwanted molecules can be separated from the target molecules. Then, the target molecules can be electrically moved to an ISFET sensor for detection. By using this idea of separating the places where DNA hybridizes and enzyme activation/signal sensing occurs, the immobilization and washing steps become unnecessary. Without the washing step, one can eliminate the huge viscous dissipation occurring in small channel. The separator constitutes a large aspect ratio channel with tens or hundreds of nanometer in one dimension. This design leads to a large increase of the sensing surface to volume ratio such that the sensitivity may be greatly enhanced.

The invention provided herein has a number of specific embodiments. An illustrative embodiment comprises integrating a biosensor (for example an ISFET (Ion Sensitive Field Effect Transistor)) into a separation channel to separate the place where target molecule/probe molecule binding occurs from the place where signal sensing. Another illustrative embodiment comprises integrating MOSFET (Metal Oxide Semiconductor Field Effect Transistor) transistors into a channel. Another illustrative embodiment comprises a 3-D MOSFET transistor which is made by fabricating the source and drain of the MOSFET on the sidewalls of the channel. In a variation on these illustrative embodiments, multiple electrodes can be integrated a channel. In a another variation on these illustrative embodiments, a dielectric material like SiO₂ can be deposited to cover the whole channel to enhance the dielectric strength of the channel.

### BRIEF DESCRIPTION OF THE FIGURES

FIG. 1 illustrates molecular beacon technology. (a) Before hybridization, the molecular beacon remains non-fluorescent because the fluorophore is quenched by the quencher, (b) molecular beacon becomes fluorescent after hybridization with targets.

FIG. 2 shows cross sections of microchannels bonded with glass (with silicon dioxide and metal layers in between). Sidewalls are coated with a metal layer to make reflection mirrors (a) rhombus channel, (b) v-groove channel. (c) trapezoid channel, (d) rectangular channel.

FIG. 3 shows microscopic pictures of channel cross-sections (a) rhombus channel, (b) v-groove channel, (c) trapezoid channel, (d) rectangular channel.

FIG. 4 shows anodic bonding of Pyrex and SiO₂ with a metal layer partially in between (a) before bonding, a space of 2200 Å between glass and SiO₂ (b) after bonding, the metal is squeezed in between.

FIG. 5 shows completely bonded channels (a) Channel is empty (b) Channel partially filled with water and no leak is observed in the Au/SiO₂ interface and top Au edges of the channel.

FIG. 6 shows a setup of a Laser Induced Fluorescence System.

FIG. 7 shows a sensor chip with 8 detection channels.

FIG. 8 shows a signal-to-noise (SNR) enhancement due to Al & Au coating for different sample concentrations.

FIG. 9 shows a comparison of SNR for different channels. The concentration of MB-DNA solution used for testing is 2 nM. Sidewalls in the detection regions of the four channels were coated with an Al layer. The channel width is 80 μm.

FIG. 10 shows SEM pictures of channels with Al coating; (a) KOH etched trapezoid channel, sidewall and bottom are smooth; and (b) DRIE etched rectangular channel, sidewall and bottom are very rough.

FIG. 11 illustrates a typical electrophoretic separator which comprises a main separation channel, a sample injector (a set of cross channels), a bio-reactor and sample/waste reservoirs.

FIG. 12 illustrates how the hybridized DNA probe and target DNA pair bears a very different mass/charge ratio compared with that of the excess DNA probes.

FIG. 13 illustrates a typical ISFET sensor which can be fabricated in the downstream of the bio-reactor. The entire channel bottom area can be deposited with pH sensitive composite thin films such as SiO₂/Si₃N₄, SiO₂/SnO2 or SiO₂/Al₂O₅ to form a gate of the ISFET and to maximize the sensing area.

FIG. 14 illustrates an embodiment of the method to detect nucleic acids and proteins wherein urease is coupled to an antibody probe.

FIG. 15 shows that for glass channels, only the photons directly emitted from the molecules are collected, thus the collection ratio is smaller than silicon channels that both direct and reflected photons are collected. 15(a) detection in a glass channel, only direct emitted light is collected, 15(b) detection in a Si channel, both direct and emitted lights are collected.

FIG. 16 provides a graphic representation (relative fluorescent intensity vs concentration) simulation results of collection ratio of emissions photons for microchannels with different geometry, sidewall coatings and substrate materials. The graph illustrates the limits for the channel with coating and without coating (1 order difference). N.A. of the lens is chosen as 0.5. The rhombus channel with both a DRIE pre-etched trench with aspect ratio of 0.06 and aluminum coating is an optimal channel design.

FIG. 17(a) provides an illustrative schematic of a molecular beacon based zepto mole sensor and 17(b) provides data from this embodiment of the invention.

FIG. 18 provides a cross-section of microchannels with 3-D electrodes for electrical molecular focusing.

FIG. 19 provides (a) a picture of the electrical focusing chip with three sensors; (b) microscopic pictures of the focusing electrodes FIG. 20 provides conceptual schematics of 3-D electrical focusing. For DNA focusing the middle is applied with positive potential and both side electrodes are grounded.

FIG. 21 provides an example of the detection of single M13 DNA bursts. (a) Blank test with TBE buffer, (b) detection without focusing, (c) detection with 225 V/cm focusing, toward the probing region (d) detection with 450 V/cm focusing toward the probing region. The DNA concentration is 20 fM. The average number of molecules in the probing volume is 0.007.

FIG. 22 provides autocorrelation functions calculated from the M13 DNA solution. (a) Unnormalized autocorrelation functions. (b) Normalized autocorrelation functions. The formula used to calculate the autocorrelation function was G(t)=(1/N)Sn(t)n(t+t), where G is the autocorrelation, N is the size of the data set, n is the value at time t, and t is the offset.

FIG. 23 shows a SEM picture of KOH etched rhombus channel with Al coating. The coating is not uniform on the sidewall which affects the SNR of detection.

FIG. 24 shows a sensitivity check of channels with different coatings. KOH etched trapezoid channels are used. Channel width and depth are 80 m and 50 m. The detection limit for Al coated channel is 7×10⁻²³ mole which is about 50 DNA molecules.

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terns with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

### I. EMBODIMENTS OF THE INVENTION COMPRISING MICROCHANNEL. BIOSENSORS FOR THE ANALYSIS OF BIOLOGICAL MOLECULES AND METHODS FOR THEIR USE

A. General Description of Reflective Microchannel. Biosensor Properties

The invention disclosed herein provides improved biosensors and assays for measuring fluorophore coupled biological molecules. By measuring the fluorescent signals of fluorophore coupled biological molecules in microchannels coated with highly reflectant metal films, the ratio of the authentic fluorophore signals to that of the noise (or background) signals as measured in an optical detection system (e.g. a Laser Induced Fluorescence (LIF) system) is increased. Specifically, by integrating such sidewall mirrors in to microchannels, the signal-to-noise ratio of the total fluorescent signal (ie. the direct fluorescent signal and the reflected fluorescent signal) of the fluorophore coupled biological molecules is increased, thereby providing a total fluorescent signal that more accurately reflects the true fluorescent status of the fluorophore coupled molecule. In an additional aspect of the invention disclosed herein, microchannels with different cross-section geometries are fabricated to optimize the design characteristics of the biosensors. As disclosed herein, geometrically distinct detection regions in channels can generate an enhanced surface reflectance and increased fluorescence signal level.

The observation that the fluorescent signal that is reflected off of the mirrored microchannels contains a higher proportion of authentic fluorophore signals as compared to spurious (“noise” of “background”) signals than does the direct (unreflected) fluorescent signal is surprising because typically reflective surfaces generate a reflected signal which is equivalent to (ie. “mrrrors”) that of a direct (unreflected) signal. Without being bound by any specific scientific theory or principle, this unexpected result could be occurring if: (1) some subset of the fluorescent noise that is directly detectable is not reflectable; and (2) there is no equivalent subset of authentic fluorescent signals.

Significant sources of noise in the unique environment generated by biosensors having microchannels are likely to include Rayleigh scattering, Raman scattering, background luminescence, and electronic noises of the photon detector. Rayleigh scattering is scattered light of the excitation wavelength. As the desired fluorescence signal will be at longer wavelengths, in principle, Rayleigh scattering can be removed by using appropriate filters. Background noise may also come from the capillary or microchannel walls and from the background impurities in the medium. The data observed herein is consistent with a paradigm where Raman scattering and fluorescence impurities in the solution as well as the authentic fluorescence signal are reflected by the mirrored channel. It is possible that the electronic noises that come from outside of the channels are reflected by the mirrored biosensors while the background luminescence that comes from the cover plate glass) that is outside the microchannel is not. As there are however, a variety of complex factors contributing to the generation of noise and the selective amplification of certain fluorescent signals within a microchannel environment, it is not possible to predict to what extent exactly what factor(s) are influencing the observed effect.

As disclosed herein, the geometry of the mirrored microchannels can be manipulated to further influence the signal-to-noise ratio of a fluorescent signal generated by a fluorophore coupled molecule within a microchannel. As it was not possible to predict how a given mirrored microchannel geometry would influence the signal-to-noise ratio of a fluorescent signal this ratio was measured in a variety of channels having different geometries. The results of these measurements are shown in FIG. 9. Surprisingly, a trapezoidal geometry provides an optimal design configuration for detection based on the currently employed fabrication techniques, with the next-most optimal design configurations being v-groove, rhombus and then rectangular geometries.

The invention disclosed herein has a number of embodiments. A preferred embodiment of the invention is a biosensor comprising a microchannel, wherein a sidewall of the microchannel has been treated or manipulated in some way so as to reflect an optical signal such as a fluorescent signal so that the signal-to-noise ratio of the reflected optical signal is increased. Throughout this application the illustrative optical signal that is discussed in detail is a fluorescence signal. Those skilled in the art will understand that this is merely provided as a representative embodiment of an optical signal that is used to a large extent in protocols designed to sense biological molecules. In this context, this aspect of the disclosure is directed to optical signals in general and is not limited to fluorescence.

In preferred embodiments the microchannel can be treated to reflect a optical signal by coating the sidewall with a reflective film of a metal such as gold or aluminum. In such embodiments, the signal-to-noise ratio of the reflected optical signal can be enhanced by about 14% to about 420% and from about 80% to about 860% respectively, for different concentrations of sample solution. While preferred embodiments of the invention contain a reflective films of gold or aluminum, skilled artisans understand that a variety of reflective materials can be used to treat a sidewall of a microchannel in order to increase the signal-to-noise ratio of a reflected optical signal.

In highly preferred embodiments of the invention, the cross-section geometrical shape of the microchannel is selected to enhance the reflected signal-to-noise ratio. In preferred embodiments, the microchannel has a cross-section geometrical shape selected from the group consisting of a rhombus, a trapezoid, a v-groove and a rectangle. In highly preferred embodiments, the microchannel has a cross-section geometrical shape that is trapezoidal. In preferred embodiments of the biosensor, the sidewall of the microchannel is constructed to aim or focus the reflected optical signal in a desired direction.

As disclosed herein, the biosensor comprising a microchannel can be fabricated by any one of a variety of techniques known in the art such as KOH etching. In addition, the biosensor with the microchannel can be fabricated on to any one of the wide variety of matrices known in the art such as a microchip. In preferred embodiments of the invention, the microchip is made of glass, silicon or plastic. While preferred embodiments of the invention are microchips made of glass, silicon or plastic, skilled artisans understand that a variety of materials are known in the art for the fabrication of microchips.

Yet another embodiment of the invention is a biosensor comprising a sensing receptacle such as a reaction chamber, channel or well, wherein a sidewall of the sensing receptacle has been treated so as to reflect an optical signal such as a fluorescence signal such that the signal-to-noise ratio of the reflected optical signal is increased. In preferred aspects of this sensor, the cross-section geometrical shape of the sensing receptacle is configured to enhance the reflected signal-to-noise ratio. In highly preferred embodiments of the invention the sidewall of the sensing receptacle is constructed to aim or focus the reflected optical signal in a desired direction.

Related embodiments of the invention include a method of measuring a fluorescence signal comprising measuring the signal of a fluorescent molecule within a microchannel, wherein a sidewall of the microchannel has been treated so as to reflect the fluorescence signal such that the signal-to-noise ratio of the reflected fluorescence signal is increased. In such methods the microchannel can be treated to reflect a fluorescence signal by coating the sidewall with a reflective film of a metal. In addition, in such methods the geometry of the microchannel can be is selected to enhance the reflected signal-to-noise ratio. In preferred embodiments of this method, the sidewall of the microchannel is able to aim or focus the reflected fluorescence signal in a desired direction. In preferred embodiments of this method, the fluorescence signal is measured by a laser induced fluorescence system.

Yet another embodiment of the invention includes a method of enhancing the optical measurement of a fluorescent signal of a fluorophore coupled to a polynucleotide or a polypeptide, the method comprising measuring the fluorescent signal of the fluorophore coupled molecule within a microchannel, wherein a sidewall of the microchannel is treated so as to reflect the fluorescence signal such that the signal-to-noise ratio of the reflected fluorescence signal is increased. In such methods the microchannel can be treated to reflect a fluorescence signal by coating the sidewall with a reflective film of a metal. In addition, in such methods the geometry of the microchannel can be is selected to enhance the reflected signal-to-noise ratio. In preferred embodiments of this method, the sidewall of the microchannel is able to aim or focus the reflected fluorescence signal in a desired direction.

In preferred embodiments of this method, the fluorophore coupled molecule is a polynucleotide, for example a molecular beacon probe having a 5′ end labeled with a fluorescein moiety and a 3′ end labeled with a fluorescein quenching moiety. In preferred embodiments of this method, the fluorescence signal is measured by a laser induced fluorescence system. In highly preferred embodiments of this method, the volume of a media having the fluorophore coupled molecule is less than about 5,000, 1000, 750, 500, 250, 100 or most preferably 50 picoliters. In a specific embodiment of this invention, the molecular beacon probe is used to detect DNA. In highly preferred embodiments of the invention, the concentration of DNA detected is less than about 1000, 100, 10, 1 or most preferably 0.1 zmol.

While the exemplary embodiments provided herein are directed to polynucleotides labelled with a fluorophore, the data presented herein provide evidence that the signal-to-noise ratio fluorescence of any macromolecule that can be labeled with fluorescence tags (e.g. polypeptides such as proteins) can be detected and enhanced using the mirrored microchannel biosensors described herein.

B. Reflective Microchannel Polynucleotide Biosensors

By using molecular beacons PB) as highly sensitive and selective nucleic acid probes, two of the major but cumbersome steps, probe immobilization and washing, of gene-based biosensors are eliminated. Molecular beacons become fluorescent only upon hybridization with target DNA/RNA molecules as the quencher is separated from the fluorophore. Moreover, using the disclosure provided herein one can enhance inchannel detection techniques. These techniques both increase the sensitivity of such assays and reduces the detection volume to about 36 pL. Consequently such techniques facilitate the integration of a biosensor into larger assays (e.g. a μ-TAS).

As disclosed herein, by integrating sidewall mirrors with microchannels, the MB signal-to-noise ratio of the optical detection of polynucleotides is increased. As shown in the examples below, microchannels coated with metal films with high reflectance can be fabricated to increase the signal level in a Laser Induced Fluorescence (LIF) system. By using the side-mirror channel with inchannel sensing technique, the concentration detection limit is 0.07 zmol with SNR=3 as a threshold. This is approximately three orders of magnitude lower than that for many other DNA detections.

Using existing technology it is possible to visualize a single light-emitting molecule but not for DNA detection with high specificity [See, e.g., W. P. Ambrose et al., Cytometry 36(1999), 224231; C. Zander et al., Chemical Physics Letters 286(1998), 45745]. Consequently the 0.07 zmol (about 50 molecules) detection limit of the sensors disclosed herein is a significant advancement in this technology. Moreover, in the case of more diluted solutions, the metallic mirror can be also used as an electrode to apply positive potential for concentrating negatively charged DNA in order to further improve the performance of the sensor.

In an additional aspect of the invention disclosed herein, microchannels with different cross-section geometries are fabricated to optimize the design characteristics of sensors used in polynucleotide detection. In this context, metals with high reflectance like Al and Au are deposited and patterned to form mirror-like sidewalls on geometrically distinct detection regions in channels to evaluate enhanced surface reflectance and increased fluorescence signal level.

If specific detection is to be performed, MB is one of the effective methods or materials that can be applied in the mirrored biosensors. However, there are other methods and materials that can be used with the sensors and methods described herein such as two probe labeling methods for specific unamplified genomic DNA detection (see, e.g. Alonso Castro[Anal. Chem. 1997, 69, 3915-3920). In such methods, two different probes are labeled on both ends of a same probe DNA molecule. The coincident detection of both dyes provides the necessary specificity of the detection. In addition, by using this mirrored microchannels for capillary electrophoresis, it is also possible to specifically detect unamplified genomic DNA molecules by comparing the patterns with the DNA ladder patterns.

Illustrative embodiments of the polynucleotide sensor and the method for using it that are disclosed herein are described in the following sections and Examples 1 and 2 below.

C. Design of Molecular Beacon Probes

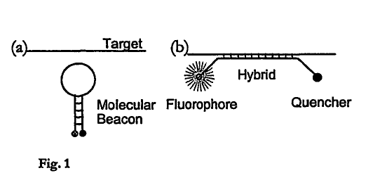

As is known in the art and illustrated in FIG. 1, molecular beacons are single stranded polynucleotide molecules with a stem-and-loop structure. The loop portion of the beacon can form a double stranded structure in the presence of its complementary nucleic acid strand. The two ends of the stems of a MB are labeled with a fluorophore and a quencher. The sequences of the two stems are typically five to eight bases long and are complementary to each other. Due to the hybridization of the two stems, the fluorophore and quencher are in close proximity to each other, causing the fluorescence to be quenched by the fluorophore (FIG. 1(a)). The sequence of the loop, which is typically twenty to thirty bases long, is designed to be complementary to sequence of the target polynucleotide molecules. In the presence of the target polynucleotide molecules, the stronger binding force between the longer loop structure and target polynucleotide will unbind the shorter/weaker stem structures and separate the quencher from the fluorophore(FIG. 1(b)).

In a specific embodiment described herein, the sequence of the loop structure is designed according to a portion of the sequence of 16 s rRNA in E. coli (MC41000) and is 22 bases long. The 5′ end is labeled with Fluorescein and the 3′ end is labeled with Dabycl quencher. The specific sequence is 5′Fluorescein-GCTCG TATTA ACTTT ACTCC CTTCC TCCGA GC-3′Dabycl (SEQ ID NO: 1).

D. Fabrication of Microchannels

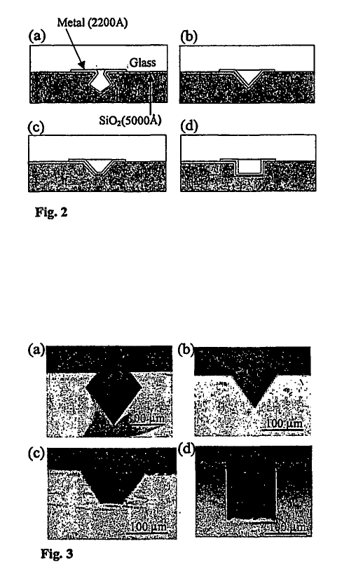

Microchannels with different geometry and metal coatings on the sidewalls can be fabricated to maximize signal-to-noise ratio. As disclosed herein, channels with v-groove, trapezoid, and rectangular cross sections were fabricated by KOH and DRIE etching, and channels with rhombus cross sections were made by DRIE pre-etching followed by KOH etching (FIGS. 2 and 3).

The typical dimensions of these mirrored channels are about 2 μm to about, 200 μm in width, about 2 μm to about 200 μm in depth and about 5 mm to about 5 cm in length. In preferred embodiments, the channel width typically varies from about 10 μm to about 150 μm and its depth typically changes from about 20 μm to about 100 μm. In a typical method for fabricating a biosensor after 5000 Å of thermal silicon oxide is grown for electrical isolation, a 2200 Å thin Al or Cr/Au layer is deposited by sputtering or e-beam evaporation. To pattern electrodes and sidewall mirrors in the deep channels, 10 μm AZP4620 PR is coated, over-exposed and developed to make etching masks or perform lift-off as the case requires.

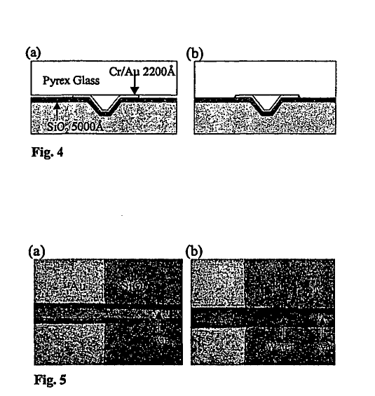

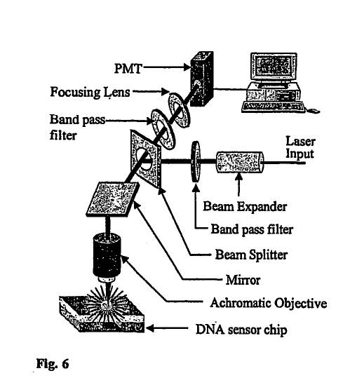

The channel chip can then be bonded to a matrix such as a pre-drilled Pyrex glass plate using for example an anodic bonding technique to form a closed channel. In spite of a spacing 2200 Å between the channel chip and the glass plate due to the metallic layer as shown in FIG. 4(a), the electric field is large enough to pull the two plates together and form a completely bonded channel. (FIG. 4(b)). Also even if the Al or Au metal layer does not bond with glass, the bonding strength between the channel chip and glass is strong enough to squeeze the metal layer tightly between those two substrates and minimize the clearance in the interface between the non-bonded metal and bonded SiO₂ areas. A complete bonded chip is then injected with water, and no leak is observed along the Au and SiO₂ interface as well as in the squeezed top metallic edges of channel. (FIG. 5).

E. Instrumentation for Micro Channel Biosensor Fluorescence Characterization

In preferred embodiments of the invention, a Laser Induced Fluorescence (LIP) system (FIG. 6) can be used for biosensor signal characterization. Typically an excitation beam (2 mW) from an air-cooled Ar ion laser (ILT, 100 mW) passes into a beam expander (Melles Griot, 09LBZ010) and a band pass filter (Omega, XF1073). It then reflects from a dichroic beam splitter (Omega, Xf2037) to a 20×0.50 N.A. objective (Rolyn Optics Company, 80.3080), which focuses the beam to a 30 μm spot within the channel. Fluorescence is collected by an objective, passes through the dichroic beam splitter, filtered by a bandpass filter (Omega, XF3003), focused by a focusing lens (Nevport, PAC052), and finally collected by a PMT (Hammatsu, HC120-01). The signal from the PMT is transmitted to a data acquisition card and typically analyzed by a Lab View program. Labview is widely as an interface for data acquisition between computer and the acquisition board. It provides user friendly GUI (Graphic User Interface) functions and a variety of interface functions for different data communication protocols. In addition to Labview, one can also use C, C++, Basic, and other computer languages for such interfacing.

II. Embodiments of the Invention Comprising Electronic Elements Integrated with Bio-Reactor/Channels

The invention disclosed herein is also directed to integrated electronics, wherein an electronic element such as a diode, a transistor, an integrated circuit etc., is integrated with a bio-reactor/channel in order to facilitating the detection and/or fabrication of bio-materials. The disclosure provided herein allows one to perform DNA, antibody or other biological molecule detection without using two of the major and necessary steps of traditional detection methods, immobilization and washing.

The invention provided herein has a number of embodiments. An illustrative embodiment comprises integrating a biosensor (for example an ISFET (Ion Sensitive Field Effect Transistor)) into a separation channel to separate the place where target molecule/probe molecule binding occurs from the place where signal sensing. Another illustrative embodiment comprises integrating MOSFET (Metal Oxide Semiconductor Field Effect Transistor) transistors into a channel. Another illustrative embodiment comprises a 3-D MOSFET transistor which is made by fabricating the source and drain of the MOSFET on the sidewalls of the channel. In a variation on these illustrative embodiments, multiple electrodes can be integrated a channel. In another variation on these illustrative embodiments, a dielectric material like SiO₂ can be deposited to cover the whole channel to enhance the dielectric strength of the channel.

The embodiments of the invention disclosed herein include an innovative approach to the detection of various macromolecules by separating the DNA hybridization/immunobiological binding process and the enzymatic reaction process for sensing into two locations by applying an electrophoretic separator. The excess enzymes and other unwanted molecules can be separated from the target molecules. Then, the target molecules can be electrically moved to an ISFET sensor for detection. By using this idea of separating the places where DNA hybridizes and enzyme activation/signal sensing occurs, the immobilization and washing steps become unnecessary. Without the washing step, one can eliminate the huge viscous dissipation occurring in small channel. The separator constitutes a large aspect ratio channel with tens or hundreds of nanometer in one dimension. This design leads to a large increase of the sensing surface to volume ratio such that the sensitivity may be greatly enhanced.

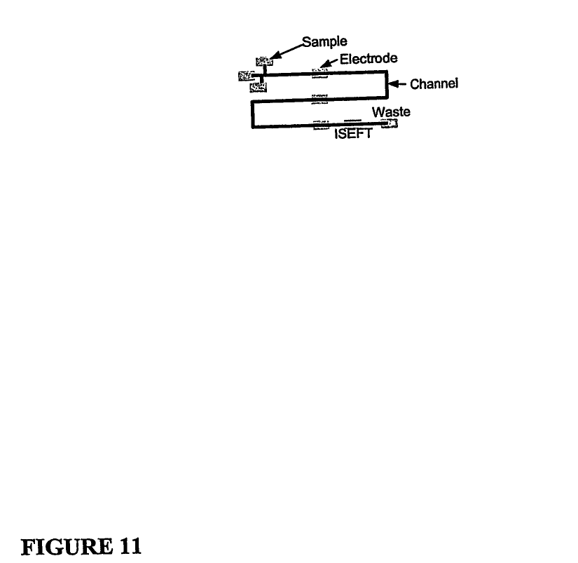

A typical electrophoretic separator (see FIG. 11) comprises a main separation channel, a sample injector (a set of cross channels), a bio-reactor and sample/waste reservoirs. A DNA sequence, which is specific for a target labeled with one of the pH-sensitive enzyme known in the art such as urease or glucose oxidase (GOD), can comprise a typical DNA probe. Samples, DNA probes and target, can be injected into the bio-reactor from the sample reservoirs by applying electrical field. If the target DNA does match with the specific sequence and can then hybridize with the DNA probe. The pH sensitive enzyme, urease, can catalyze the substrate solution containing urea or hydrogen peroxide and then change the local pH of the buffer solution (e.g. CO(NH₂)₂+3H₂O CO₂+2NH₄⁺+2OH⁻).

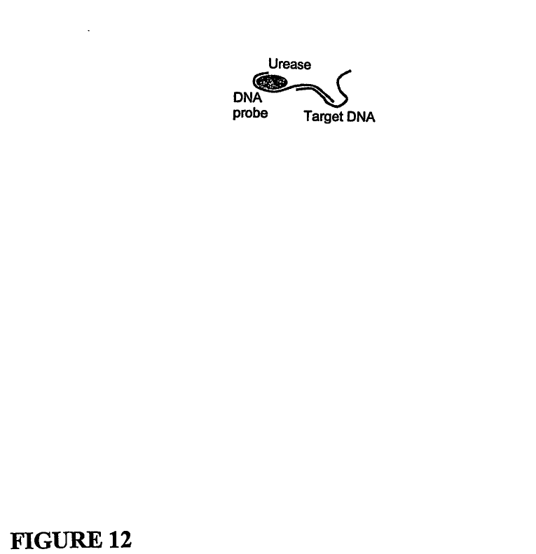

As illustrated by FIG. 12, the hybridized DNA probe and target DNA pair bears a very different mass/charge ratio compared with that of the excess DNA probes. The mixed molecules in the bio-reactor can be separated into two bands after transported by electrophoretic forces through the nano channel separator. The typical dimension of the channels that can be used in these embodiments are about 0.5 μm to about 50 μm in width, about 0.05 μm to about 10 μm in depth and about 5 mm to about 5 cm in length. In preferred embodiments the dimension of the channel-FET gate area is designed at about 50 μm in length and about 100 μm in width. The depth of the channel can be about 500 nanometer or less. The width can be in the order of about 100 microns. The surface to volume ratio can be much larger than that of a smaller aspect ratio channel with the same cross-section area; thereby enhancing the sensor sensitivity.

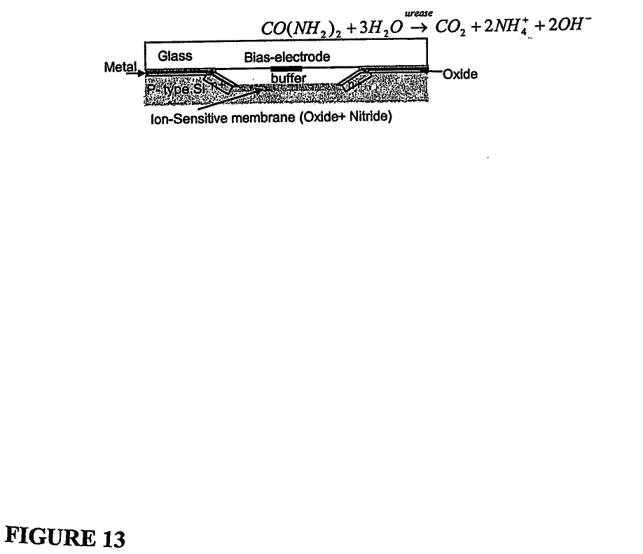

As illustrated by FIG. 13, an ISFET sensor can be fabricated in the downstream of the bio-reactor. The entire channel bottom area can be deposited with pH sensitive composite thin Elms such as SiO₂/Si₃N₄, SiO₂/SnO₂ or SiO₂/Al₂O₅ to form a gate of the ISFET and to maximize the sensing area. The source and drain of the ISFET can be doped with n-type or p-type dopants in the side walls. The source and drain areas can be covered by an oxide layer and their contact windows can be defined and deposited with W/Ti metals. Prior to bonding with a glass or PDMS plate the chip can be further covered by a passivation oxide layer, and planarized by a CMP process. This ISFET bio-sensor can significantly reduce the sample volume needed for detection and increase the sensitivity by virtue of its large surface to volume ratio. The operational principle for the ISFET is based the reaction of the hydrogen or hydroxyl ions with the ion sensitive gate thin films.

These reactions are (using SiO₂ as an example):
SiOH<==>SiO⁻+H⁺ and SiOH+H⁺<==>SiOH₂⁺
The surface of gate oxide is either exposed or blocked by the formation of SiOH. The response of pH-sensitive layer can be as high as about 40-60 mV pH⁻¹ and be linear between about pH 2 and about pH 10.

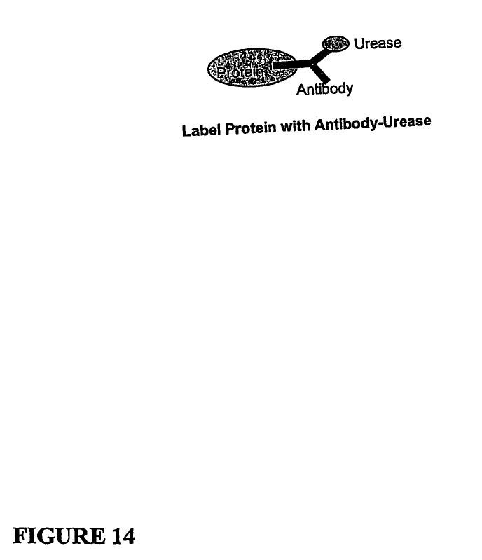

While the molecules bound with enzymes flowing by the ISFET integration area, the pH variation can be sensed by the ISFET. Thus, unknown DNA molecules can be identified and detected. If the target DNA hybridizes with the sequence specific probe, the ISFET located downstream of the bioreactor can first detect the band containing the excess unhybridized probes. It can then sense the passing of another band of hybridized target DNA and the probe labeled with enzymes. If the target DNA strand does not match with the probe sequence, the sensor can only detect the passing of the unhybridized DNA probes. This unique DNA sensor does not require immobilization. Similar method can be implemented to detect protein by labeling urease to the antibody probe instead (see FIG. 14).

The invention disclosed herein has a number of embodiments. A typical embodiment of the invention is a biosensor comprising a sensing receptacle such as a channel or a chamber or a well in which a target molecule and a probe for the target molecule interact, wherein the sensing receptacle is integrated with an electronic element selected from the group consisting of a transistor, a diode and an integrated circuit. In preferred embodiments, the electronic element is selected from the group consisting of an ion sensitive field effect transistor and a metal oxide semiconductor field effect transistor. In preferred embodiments of the invention, the sensing receptacle is a microchannel. Such embodiments include sensors wherein two or more electrodes are integrated into the microchannel. Optionally the dielectric strength of the microchannel is enhanced by including a dielectric material within the channel, typically SiO₂. In a specific aspect of this embodiment, the electronic element is a metal oxide semiconductor field effect transistor comprising a source and a drain fabricated so that the source and drain of the metal oxide semiconductor field effect transistor are on the sidewalls of the microchannel.

Yet another embodiment of the invention is a biosensor comprising a sensing receptacle, wherein sidewalls and bottom of the sensing receptacle have been treated as discrete electrodes so as to electrically concentrate molecules in a certain region in a manner that enhances the detection efficiency of the biosensor. Typically the electrodes in a receptacle can be made by coating and patterning with a metal such as aluminum or gold. In this context, the use of metals such as gold can increase the detection efficiency by at least about 500%. As further described herein, the receptacle is typically in a microchip which is made from one of the materials commonly used in this art such as silicon, glass or plastic.

Another embodiment of the invention is a method for detecting a target molecule selected from the group consisting of a polypeptide and a polynucleotide by allowing the target molecule and a probe for the target molecule to interact within a first area on a biosensor comprising an ion sensitive field effect transistor sensor and a separation channel, moving the target molecule and the probe for the target molecule that have interacted to a second area on the biosensor through the separation channel via electrophoresis, and then sensing a signal generated by the interacted target molecule and the probe for the target molecule in the second area of the biosensor via the ion sensitive field effect transistor sensor. In a preferred embodiment, the separation channel is a microchannel.

Another embodiment of the invention is an electrophoretic separator comprising a target molecule sample injector comprising a set of cross channels, a sample reservoir, a sensing receptacle such as a channel or a chamber or a reservoir constructed to allow the interaction between a target molecule and a probe for the target molecule, a separation channel, and a waste receptacle. In preferred aspects of this embodiment, the separator comprises a large aspect ratio channel of tens to hundreds of nanometers in one direction. In highly preferred embodiments, the separation channel is a microchannel. In certain embodiments of the invention, this electrophoretic separator is constructed so that the a target molecule and a probe for the target molecule can be introduced into the sensing receptacle from the sample reservoir by applying an electrical filed to the electrophoretic separator. Optionally the separation channel in this separator further comprises a pH sensitive composite thin film.

The invention disclosed herein provides significant advantages over existing inventions and is the only method to make a bio-sensor using DNA hybridization or immunoassay methods without probe immobilization and debris washing steps. Moreover, the minimum detectable number of molecules is much smaller than the state of the art, in other words, it can be more sensitive. In addition, this invention can easily be integrated in a lab-chip or micro total analysis system (μ-TAS) system.

As noted above, the inventions disclosed herein have a number of embodiments. In a further embodiment of the invention, there are provided articles of manufacture and kits containing materials useful for the analysis of biological molecules. The article of manufacture comprises a container with a label. Suitable containers include, for example, a biosensor having a microchannel wherein a sidewall of the microchannel has been treated so as to reflect a fluorescence signal such that the signal-to-noise ratio of the reflected fluorescence signal is increased. The containers may be formed from a variety of materials such as glass or plastic. The label on the container can indicates that the biosensor is used in methods for analyzing fluorescenated molecules, such as those described above.

Throughout this application, various publications are referenced. The disclosures of these publications are hereby incorporated by reference herein in their entireties. The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### EXAMPLES

### Example 1

### Microchannels Integrated with Sidewall Mirrors for Biological Detection Using Molecular Beacon Technology

This illustrative example discloses the fabrication of microchannels integrated with sidewall mirrors and the application for in-channel optical DNA/RNA detection. The detection specificity is achieved by using molecular beacon CB) based DNA hybridization technique. Molecular beacons are highly sensitive and selective oligonucleotide probes (see e.g. S. Tyagi, F. R Kramer, “Molecular beacons: Probes that fluoresce upon hybridization” Nature Biotechnol. 14 (1996), 303-308) that become fluorescent upon hybridization with target DNA/RNA molecules as shown in FIG. 1. By using MB probe technology, two of the major but cumbersome steps of gene-based biosensors, probe immobilization and washing (see e.g. S. R. Mikkelsen, Electroanalysis, 8 (1996) 15; J. Gau, E. Lan, et al., Proceedings of the Fourth International Symposium on I-TAS (2000)), can be eliminated. This realizes in-channel detection and reduces the detection volume to about 45 pl which is at least about 3 order of magnitude reduction than many other DNA detection (see e.g. X. Liu, W. Farmerie, et al., Anal Biochem. 283(2000), 56-63; M. Yang, M. E. McGovern et al., Analytica Chimica Acta, 346(1997), 259-275).

Microchannels are fabricated by KOH etching to make smooth sidewalls and are coated with metal films with high reflectance to improve the reflectivity, resulting the enhancement of detection sensitivity. The concentration detection limit of the channel for its targets is about 0.02 nM which is approximately two orders of magnitude lower than that for many other DNA detection (see e.g. M. Yang, M. E. McGovern, et al., Analytica Chimica Acta, 346(1997), 259-275).

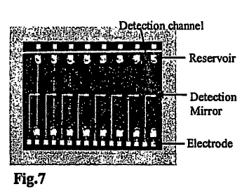

Microchannels with different substrate maters (e.g. glass, silicon), geometry, and metal coatings on the side-walls of detection regions are fabricated and compared to determine the optimum design for detection. Channels with rectangular, v-groove and trapezoid, and rhombus cross sections as shown in FIG. 2 are fabricated by DRIE etching, KOH etching, and DRIE etching following by KOH etching respectively. The width and depth of channels vary from about 10 μm to about 150 μm and about 20 μm to about 100 μm. After 5000 Å of thermal silicon oxide is grown for electrical isolation, a thin Al or Cr/Au layer of 2200 Å is deposited by sputtering or e-beam evaporation. To pattern electrodes and sidewall mirrors on the deep channels, 10 μm thick PR is over-exposed and developed for making etching masks or performing lift-off technique. The channel chips are then bonded with a pre-drilled Pyrex glass by anodic bonding. FIG. 3 shows the microscopic pictures of different cross-sections of channels and FIG. 7 shows a detection channel chip with 8 channels on it.

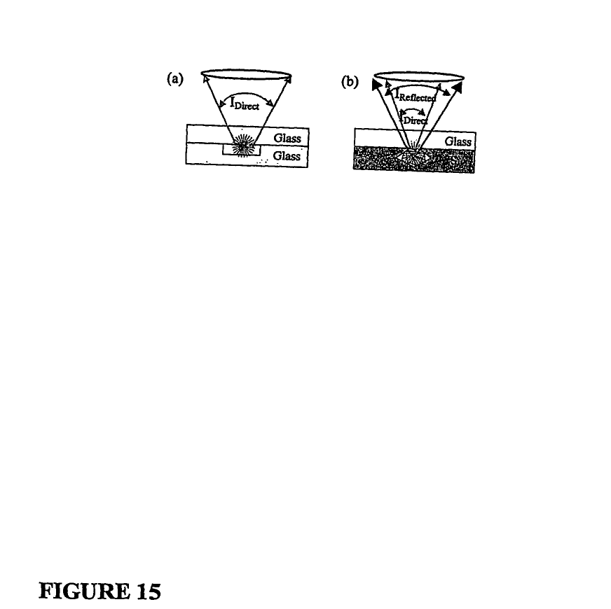

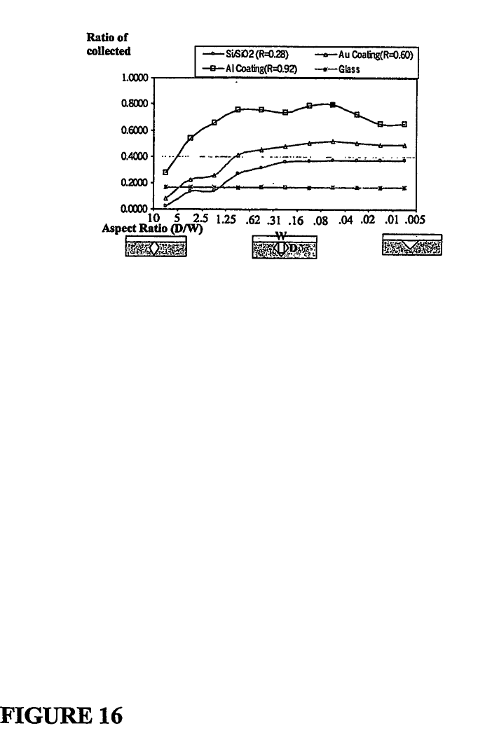

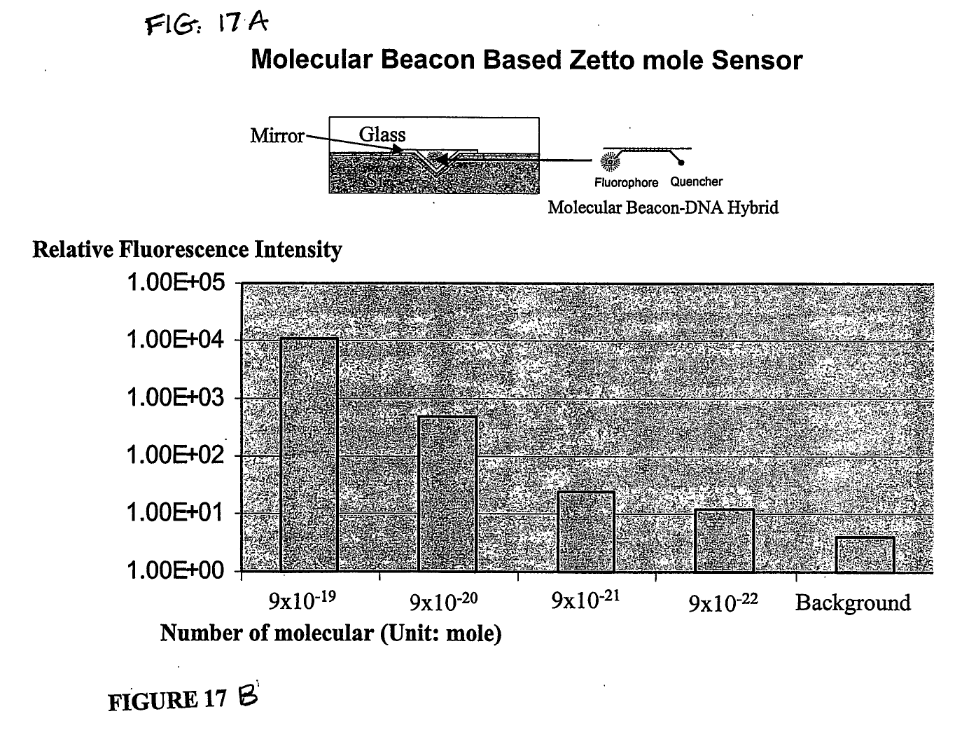

The ratio of emitted photons collected by an objective lens from the MB/DNA hybrids is a function of numerical aperture of the lens, substrate materials, channel geometry, surface roughness and surface coating. A simulation program is written to compare the photon collection ratio for the different channels. For glass channels, only the photons directly emitted from the molecules are collected, thus the collection ratio is smaller than silicon channels that both direct and reflected photons are collected as shown in FIG. 15. The simulation results shown in FIG. 16 also shows that the rhombus channel with both a DRIE pre-etched trench with aspect ratio of about 0.06 and aluminum coating is an optimal design. The ratio is 2.2 times higher than the channel without coating and 4.7 times higher than glass channel.

Fluorescent measurements can be performed using a Laser Induced Fluorescence (LIF) system. With the emission wavelength of 512 nm, it is found that the v-groove channel coated with aluminum has the highest SNR for the same target concentration and is 3.8 times higher than that without coating. By using this channel the detection limit is 0.02 nM (with SNR=3) and 0.90 zmol. The experimental data shows that the optimum designed channel. (rhombus cross-section, aspect ratio 0.06) does not have the best sensitivity because of inconsistent uniformity of coating for this type of channels, which affects the reflectance. Also because of the negative charge property of DNA molecules, positive potential is applied to the coated mirror region to confine molecules to the detection area. With this scheme, an initial polynucleotide concentration 10 times lower than the typical detection limit can be detected.

### Example 2

### Microchannels of Different Geometries Integrated with Sidewall Mirrors for Polynucleotide Detection Using Molecular Beacons

In this example, the eight hundred bases long nucleic acid targets used for detection were synthesized by polymerase chain reaction (PCR). The sense (5′-CAGAT GGGAT TAGCT AGTAG GTG-3′) (SEQ ID NO: 2) and antisense (5′-GTCTC ACGGT TCCCG AAGGC AC-3) (SEQ ID NO: 3) primers derived from the most conserved region of 16s rRNA of E. coli. (MC41000) were identical to those used in the previously reported study [See, e.g., T. H Wang et al., proceedings of METMBS'00, pp295-300]. Initially, 50 μl DNA solution with concentration of 0.2 μM and 50 μl MB solution of the same concentration were mixed for biosensor characterization. The signal to noise ratio (SNR) and detection limit of channels with various geometries and surface coatings are determined by testing the different channels with the serially diluted solution. Because of the introduction of molecular beacons, two of the major but cumbersome steps, immobilization and washing, for typical DNA hybridization technique were eliminated. This greatly simplified the preparation steps for DNA detection. The mixed solution was pipetted into the inlet of the drilled glass hole on the sensor chip (FIG. 7), and the surface tension force pulls the solution to the coated detection region. A 488 nm light beam form the Ar ion laser was focused onto the coated region, and the existence of target DNA can be identified by checking the intensity of the emitted fluorescence light, with λ=512 nm, which comes from stretched Fluorescein labeled MB probes.

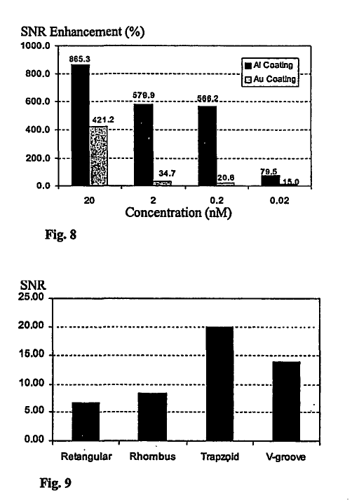

For the same geometry of microchannels, those with Al coating in the detection region was found to have the highest signal to noise ratio over those coated with Au or SiO₂ in the same detection condition. In addition, microchannels with Au coating has higher SNR than those without any metallic coating which is SiO₂ surface. The SNR enhancement for the Al and Au coated channels over SiO₂ coated channels ranges from 80% to 860% and from 14% to 420% respectively for different concentrations of sample solution as shown in FIG. 8. It is believed that the enhancement is because of the improvement of reflectance which was due to the metallic coatings. In the wavelength range of emitted fluorescence in the experiment which was 512 nm in air (385 nm in aqueous solution), the average reflectance (normal incidence) for Al thin film is 0.92, for Au thin film is about 0.40, and for SiO₂ thin film is less 0.20 [See, e.g., M. Bass, “Handbook of Optics”, 2^{nd} Edition, V.II, Mc-Graw Hill]. The magnitude order of reflectance agrees with the experimental SNR enhancement data.

Comparing the SNR of rectangular, V-groove, trapezoid, and rhombus channels, it is found that SNR of the trapezoid channel is 3.0 times higher than that of rectangular channel, and is 2.4 times higher than that of rhombus channel. (FIG. 9). The experiments are performed by injecting 2 nM sample solution (NA-MB hybridization products) into the various channels with the same width of 80 μm.

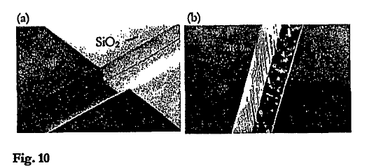

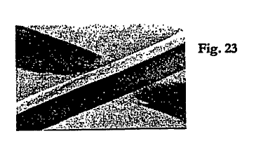

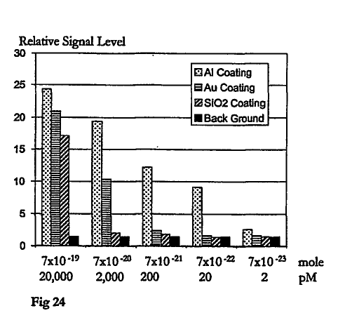

Channels fabricated by KOH etching have much smoother sidewalls than those made by DRIE etching (FIG. 10(a), (b)), and have higher overall reflectivity for the same surface coating condition. For the KOH etched channels, due to the deposition and patterning difficulties in creating a uniform metal coating on the four sidewalls of the rhombus channels (FIG. 23), the SNR of the rhombus channels is lower than those of trapezoid and V-groove channels.

As shown above, a trapezoid channel is an optimal design for use in polynucleotide detection with current fabrication techniques. Thus, trapezoid channels coated with Al, Au, and thermal oxide were tested to compare the detection limits. As shown in FIG. 24, the detection limit for a channel with thermal oxide coating is 200 pM while for a channel with Au coating is 20 pM. For an Al coated channel, the detection limit is as low as 2 pM which is about only 0.07 zmol (0.07×10⁻²¹ mole, about 50 DNA molecules) in the 36 pL probe volume (based on a 30 μm dia. focusing spot and 50 μm channel depth). This is approximately three orders of magnitude lower than that for many other DNA detections [See, e.g., M. Yang, et al., Analytica Chimica Acta, 346(1997), 259-275; D. Ivnitski et al., Biosensors & Bioelectronics 14 (1999), 599-624].

### Example 3

### Electrical Focusing for Laser Induced Fluorescence Based Single DNA Molecules Detection

This example describes a method, 3-D electrokinetic focusing technique, to concentrate fluorescence labeled molecules into a tiny probing volume to enhance the mass detection efficiency for laser induced fluorescence (LIF) based molecular sensing. By applying this method for detecting DNA of very low concentration (20 fM), single molecule fluorescence bursts were real time determined, and more than five times enhancement of mass detection efficiency was achieved. Comparing this method with the other molecular focusing techniques such as hydrodynamic focusing [A. Castro and J. G. K Williams, Anal. Chem. 69, 3915-3920 (1997)] and electric current focusing [S. C. Jacobson and J. M. Ramsey, Anal. Chem. 69, 3212-3217 (1997)]. This method can also enhance the concentration detection limit and overcome the off-center problems of sample stream due to the slight conductivity changes of the cross channels.

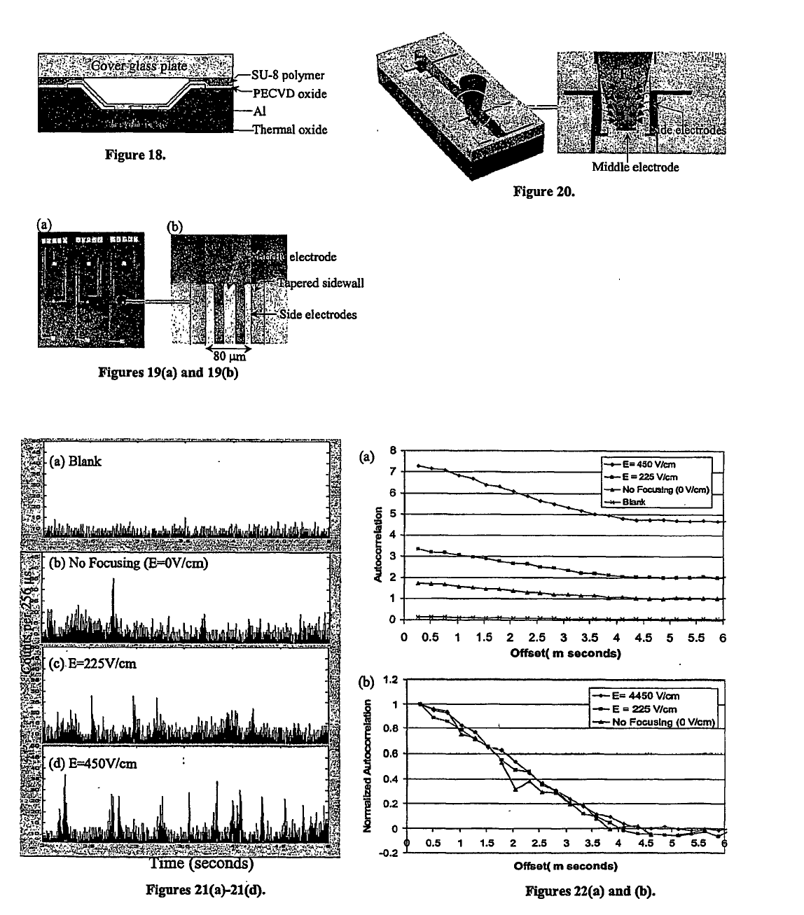

Microchannels were fabricated on silicon substrate by KOH etching to have smooth and tapered sidewalls for better metallic coverage(FIG. 18). After 5000 Å of thermal silicon oxide is grown for electrical isolation, a thin Al layer of 2000 Å is deposited by e-beam evaporation. To pattern 3-D electrodes on top of the channel sidewall and bottom, 10 μm thick PR is over-exposed and developed for lift-off technique [T. H. Wang, S. Masset, and C. M. Ho, MEMS 2001]. Another oxide layer of 5000 Å is deposited and patterned to cover the electrode to prevent the generation of bubbles due to electrolysis. The channel chips (FIG. 19) were then bonded with a pre-drilled Pyrex glass plate by using UV-curable Polymer (SU-8) bonding.

While DNA molecules passing the LIF probing volume they are electrically moved along the electrical fields and are concentrated to the bottom electrode for detection (FIG. 20). Since the molecules are precisely focused on top of the middle electrode where is designed as the focal region of the LIF, the molecule passing events can be more efficiently measured with mote consistent signal level.

Highly diluted solution of M13 DNA (7250 bp) stained with YOYO-1 fluorescence dyes was used for the preliminary tests. When DNA solutions were introduced, discrete fluorescence bursts were seen due to the passage of individual DNA molecules through focused laser beam (FIG. 20). The probability of more than one DNA molecule simultaneously occupying the probe volume can be calculated according the concentration (20 fM) and the probing volume of the implemented confocal LIF (0.6 pL). Since the calculated probability is as low as 0.007 the observed fluorescence bursts can be attributed to single molecules of DNA [K Peck, L. Stryer, A. N. Glazer, and R. A. Mathies, Proc. Natl. Acad. Sci. USA 86, 4087-4091 (1989)].

As shown in FIG. 21(b), the single molecule bursts were rarely observed without applying the focusing. After the electrical fields were applied to focus the DNA molecules to the probing region, the frequency of the single molecule bursts was increased and was proportional to the magnitude of the applied fields (FIG. 21(c), 5(d)). As a result the mass detection efficiency was enhanced. The autocorrelation function was calculated to demonstrate the presence of non-Poissonian bursts due to single molecules and in characterizing transit times (FIG. 22). The unnormalized autocorrelations in FIG. 22(a) shows that the magnitude of the autocorrelations increases with the applied electrical focusing fields, and the normalized autocorrelations in FIG. 22(b) shows that the shape and width of the autocorrelation functions change very little with the fields, as expected for single molecule detection.

1. A biosensor comprising a microchannel, wherein a sidewall of the microchannel has been treated so as to reflect a optical signal such that the signal-to-noise ratio of the reflected optical signal is increased.

2. The biosensor of claim 1, wherein the microchannel has been treated so as to reflect a optical signal by coating the sidewall with a reflective film of aluminum.

3. The biosensor of claim 2, wherein the signal-to-noise ratio of the reflected optical signal is enhanced by at least about 80%.

4. The biosensor of claim 1, wherein the microchannel has been treated so as to reflect a optical signal by coating the sidewall with a reflective film of gold.

5. The biosensor of claim 4, wherein the signal-to-noise ratio of the reflected optical signal is enhanced by at least about 15%.

6. The biosensor of claim 1, wherein the microchannel has a cross-section geometrical shape selected from the group consisting of a rhombus, a trapezoid, a v-groove and a rectangle.

7. The biosensor of claim 6 wherein the microchannel cross-section geometrical shape is a trapezoid.

8. The biosensor of claim 1, wherein the microchannel is fabricated by KOH etching.

9. The biosensor of claim 1, wherein the microchannel is in a microchip comprising a material selected from silicon, glass or plastic.

10. A method of measuring a fluorescence signal comprising measuring the signal of a fluorescent molecule within a microchannel, wherein a sidewall of the microchannel has been treated so as to reflect the fluorescence signal such that the signal-to-noise ratio of the reflected fluorescence signal is increased.

11. The method of claim 10, wherein the sidewall of the microchannel has been treated so as to reflect a fluorescence signal by coating the sidewall with a reflective film of aluminum.

12. The method of claim 11, wherein the signal-to-noise ratio of the reflected fluorescence signal is enhanced by at least about 80%.

13. The method of claim 10, wherein the microchannel has been treated so as to reflect a fluorescence signal by coating the sidewall with a reflective film of gold.

14. The method of claim 13, wherein the signal-to-noise ratio of the reflected fluorescence signal is enhanced by at least about 15%.

15. The method of claim 10, further comprising selecting a cross-section geometrical shape for the microchannel that enhances the reflected signal-to-noise ratio.

16. The method of claim 10, wherein the microchannel has a cross-section geometrical shape selected from the group consisting of a rhombus, a trapezoid, a v-groove and a rectangle.

17. The method of claim 16 wherein the microchannel cross-section geometrical shape is a trapezoid.

18. The method of claim 10, wherein the fluorescence signal is measured by a laser induced fluorescence system.

19. The method of claim 10, wherein the fluorescent molecule comprises a polynucleotide coupled to a fluorescein moiety.

20. A method of enhancing the optical measurement of a fluorescent signal of a fluorophore coupled to a molecule selected from the group consisting of a polynucleotide and a polypeptide, the method comprising measuring the fluorescent signal of the fluorophore coupled molecule within a microchannel, wherein a sidewall of the microchannel is treated so as to reflect the fluorescence signal such that the signal-to-noise ratio of the reflected fluorescence signal is increased.

21. The method of claim 20 wherein the fluorophore coupled molecule is a polynucleotide.

22. The method of claim 21 wherein the fluorophore coupled polynucleotide comprises a molecular beacon probe having a 5′ end labeled with a fluorescein moiety and a 3′ end labeled with a fluorescein quenching moiety.

23. The method of claim 20, wherein the sidewall of the microchannel has been treated so as to reflect a fluorescence signal by coating the sidewall with a reflective film of aluminum.

24. The method of claim 23, wherein the signal-to-noise ratio of the reflected fluorescence signal is enhanced by at least about 80%.

25. The method of claim 20, wherein the microchannel has been treated so as to reflect a fluorescence signal by coating the sidewall with a reflective film of gold.

26. The method of claim 26, wherein the signal-to-noise ratio of the reflected fluorescence signal is enhanced by at least about 15%.

27. The method of claim 20, wherein the microchannel has a cross-section geometrical shape selected from the group consisting of a rhombus, a trapezoid, a v-groove and a rectangle.

28. The method of claim 27 wherein the microchannel cross-section geometrical shape is a trapezoid.

29. The method of claim 20, wherein the fluorescence signal is measured by a laser induced fluorescence system.

30. The method of claim 20, wherein the volume of a media having the fluorophore coupled molecule is less than about 50 picoliters.

30. The method of claim 22, wherein the molecular beacon probe is used to detect DNA.

31. The method of claim 30, wherein the concentration of DNA detected is less than about 0.1 zmol.

32. The sensor of claim 1, wherein the sidewall of the microchannel is constructed to aim or focus the reflected optical signal in a desired direction.

33. The method of claim 10, wherein the sidewall of the microchannel is able to aim or focus the reflected fluorescence signal in a desired direction.

34. The method of claim 20, wherein the sidewall of the microchannel is configured to aim or focus the reflected fluorescence signal in a desired direction.

35. A biosensor comprising a sensing receptacle, wherein a sidewall of the sensing receptacle has been treated so as to reflect a optical signal such that the signal-to-noise ratio of the reflected fluorescence signal is increased.

36. The biosensor of claim 35, wherein a cross-section geometrical shape of the sensing receptacle is configured to enhance the reflected signal-to-noise ratio.

37. The method of claim 35, wherein the sidewall of the sensing receptacle is constructed to aim or focus the reflected optical signal in a desired direction.

38. A biosensor or chemical sensor comprising a sensing receptacle in which a target molecule and a probe for the target molecule interact, wherein the sensing receptacle is integrated with an electronic element selected from the group consisting of a transistor, a diode and an integrated circuit.

39. The biosensor or chemical sensor of claim 38, wherein the electronic element is selected from the group consisting of an ion sensitive field effect transistor and a metal oxide semiconductor field effect transistor.

40. The biosensor or chemical sensor of claim 38, wherein the sensing receptacle is a microchannel.

41. The biosensor or chemical sensor of claim 40, wherein two or more electrodes are integrated into the microchannel.

42. The biosensor or chemical sensor of claim 40, wherein the dielectric strength of the microchannel is enhanced by including a dielectric material within the channel.

43. The biosensor or chemical sensor of claim 42, wherein the dielectric material is SiO₂.

44. The biosensor or chemical sensor of claim 40, wherein the electronic element is a metal oxide semiconductor field effect transistor comprising a source and a drain fabricated so that the source and drain of the metal oxide semiconductor field effect transistor are on the sidewalls of the microchannel.

45. A method for detecting a target molecule selected from the group consisting of a polypeptide and a polynucleotide comprising the steps of:
(a) allowing the target molecule and a probe for the target molecule to interact within a first area on a biosensor comprising an ion sensitive field effect transistor sensor and a separation channel;
(b) moving the target molecule and the probe for the target molecule that have interacted to a second area on the biosensor through the separation channel via electrophoresis; and
(c) sensing a signal generated by the interacted target molecule and the probe for the target molecule in the second area of the biosensor via the ion sensitive field effect transistor sensor.

46. The method of claim 45, wherein the separation channel is a microchannel.

47. A biosensor comprising a sensing receptacle, wherein sidewalls and bottom of the sensing receptacle have been treated to function as discrete electrodes capable of electrically concentrating a molecule in a predetermined region of the biosensor.

48. The biosensor of claim 47, wherein electrodes are made by coating and patterning the receptacle with aluminum.

49. The biosensor of claim 47, wherein electrodes are made by coating and patterning the receptacle with gold.

50. The biosensor of claim 48, wherein the electrodes increase the biosensor's sensitivity for the detection a molecule by at least about 500%.

51. The biosensor of claim 47, wherein the receptacle is in a microchip comprising a material selected from the group consisting of silicon, glass and plastic.
